(19) United States
(12) Patent Application Publication
(10) Pub. No.: US 2021/0031007 A1
(43) Pub. Date: Feb. 4, 2021

(54) Fluidic Drivers, Devices, Methods, and Systems for Catheters and Other Uses

(71) Applicant: Project Moray, Inc., Belmont, CA (US)

(72) Inventors: Miles D. Alexander, Fremont, CA (US); Keith Phillip Laby, Oakland, CA (US); Ronald G. Williams, Menlo Park, CA (US)

(73) Assignee: Project Moray, Inc., Belmont, CA (US)

(21) Appl. No.: 17/064,182

(22) Filed: Oct. 6, 2020

Related U.S. Application Data
(63) Continuation of application No. PCT/US2019/026360, filed on Apr. 8, 2019.
(60) Provisional application No. 62/654,092, filed on Apr. 6, 2018.

(51) Int. Cl. A61M 25/01; A61B 34/30; A61B 46/13

(52) U.S. Cl. CPC A61M 25/0155; A61B 46/13; A61B 34/30

(57) ABSTRACT

Devices, systems, and methods can articulate catheters and other tools using fluid drive systems that provide robotically coordinated motion. The drive power will often be transmitted from a fluidic driver to the catheter through a series of pneumatic or hydraulic channels and the driver can be isolated from a sterile field by encasing the driver in a sterile housing and directing drive fluid through a sterile junction between the catheter and driver. Interventional physicians can retain tactile feedback by manually advancing the catheter over a wire or the like, and can subsequently bring an interface of the catheter down into engagement with a corresponding driver interface once a therapeutic tool approaches a target site. A sensor may provide signals during manual advancement of the driver and catheter along the catheter axis.

### CROSS REFERENCE TO RELATED APPLICATIONS

The present application is a Continuation of PCT/US2019/026360 filed Apr. 8, 2019; which claims the benefit of U.S. Provisional Appln No. 62/654,092 filed Apr. 6, 2018; the full disclosures which are incorporated herein by reference in their entirety for all purposes.

### FIELD OF THE INVENTION

In general, the present invention provides improved devices, systems, and methods for use in articulating elongate flexible bodies and other tools such as catheters, borescopes, continuum robotic manipulators, and the like. In some exemplary embodiments, the invention provides hydraulic or pneumatic drive structures for altering the shape of catheters, particularly for those catheters having an articulation balloon array in which subsets of balloons can be selectively and variably inflated to bias the catheter to bend, elongate, and/or deploy a therapeutic device within a patient.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging, in-part due to the difficulty in accessing (and aligning with) a target tissue using an instrument traversing tortuous vasculature. Alternative minimally invasive surgical technologies include robotic surgery, and robotic systems for manipulation of flexible catheter bodies from outside the patient have also previously been proposed. Some of those prior robotic catheter systems have met with challenges, possibly because of the difficulties in effectively integrating large and complex robotic pull-wire catheter systems into the practice of interventional cardiology as it is currently performed in clinical catheter labs. While the potential improvements to surgical accuracy make these efforts alluring, the capital equipment costs and overall burden to the healthcare system of these large, specialized systems is also a concern. Examples of prior robotic disadvantages that would be beneficial to avoid may include longer setup and overall procedure times, deleterious changes in operative modality (such as a decrease in effective tactile feedback when initially accessing or advancing tools toward an internal treatment site), and the like.

A new technology for controlling the shape of catheters has recently been proposed which may present significant advantages over pull-wires and other known catheter articulation systems. As more fully explained in US Patent Publication No. 20160279388, entitled “Articulation Systems, Devices, and Methods for Catheters and Other Uses,” published on Sep. 29, 2016 (assigned to the assignee of the subject application and the full disclosure of which is incorporated herein by reference), an articulation balloon array can include subsets of balloons that can be inflated to selectively bend, elongate, or stiffen segments of a catheter. These articulation systems can direct pressure from a simple fluid source (such as a pre-pressurized canister) toward a subset of articulation balloons disposed along segment(s) of the catheter inside the patient so as to induce a desired change in shape. These new technologies may provide catheter control beyond what was previously available, often without having to resort to a complex robotic gantry, without having to rely on pull-wires, and even without having the expense of motors. Hence, these new fluid-driven catheter systems appear to provide significant advantages.

Despite the advantages of the newly proposed fluid-driven robotic catheter systems, as with all successes, still further improvements and alternatives would be desirable. In general, it would be beneficial to provide further improved medical devices, systems, and methods, as well as to provide alternative devices, systems, and methods for other robotic tools. More specifically, it may be beneficial to provide new technologies to maintain a sterile field encompassing an access site used for treatment of a target tissue, ideally without having to subject a fluidic drive system to repeated deleterious sterilization. It would be particularly beneficial to avoid any need to resort to complex articulated sterile housing assemblies, and if these new technologies could be used safely for a particular patient without having the delay and disruption of bagging an articulatable robotic arm in a series of sterile drapes. It may also be beneficial to provide improved fluidic drive structures and methods appropriate for use with robotic surgical tools and other uses.

### BRIEF SUMMARY OF THE INVENTION

The present invention generally provides improved devices, systems, and methods for articulating elongate flexible structures such as catheters, borescopes, continuum robotic manipulators, and the like. The elongate flexible structures described herein will often include an array of fluid-expandable bodies such as balloons, and fluid drive systems described herein will often be used to drive these catheters (or other structures) with robotically coordinated motion. Hence, the drive structures described herein will optionally allow the user to position and orient a therapeutic tool in a beating heart of a patient without having to individually determine articulated segment shapes or individual joint configurations along the axis of the catheter. As the drive power will often be transmitted from a fluidic driver to the catheter through a series of pneumatic or hydraulic channels, the driver can be isolated from the sterile field by encasing the driver in a sterile housing during use, with the drive fluid passing through a sterile barrier of a sterile junction between the catheter and driver. Interventional physicians can retain tactile feedback associated with advancing of known catheter-supported tools toward a target tissue by manually advancing the catheter over a wire or the like. After a therapeutic tool approaches a target site, the user can then mount an interface of the advanced catheter onto a corresponding driver interface for robotic articulations. A sensor may provide signals during any final manual advancing of the driver and catheter together along the catheter axis, with elongation of a distal articulated portion of the catheter optionally providing axial fine-tuning of the therapeutic tool alignment. Several other improvements and refinements are also provided herein.

In a first aspect, the invention provides a robotic system for treating a patient. The patient has a tissue accessible from within a sterile field, and the system comprises an actuated tool having a proximal tool interface and a distal portion configured for alignment with the tissue. An actuatable feature is disposed along the distal portion of the tool, the actuatable feature being operatively coupled with the tool interface. A driver of the system has a fluid supply configured to drive the tool, and also has a driver interface. A sterile housing fittingly receives the driver, the sterile housing including a sterile junction having a sterile barrier extendable between the tool interface and the driver interface so that the fluid supply can drive the actuatable feature through the sterile housing when the tool interface is coupled with the driver interface. The sterile housing has an outer surface and is configured to maintain sterile separation between the sterile field (adjacent the outer surface) and the driver (within the sterile housing) when the robotic surgical system is configured for use.

In another aspect, the invention provides a sterile structure for use in a robotic surgical system for treating a patient. The patient has a tissue accessible from within a sterile field, and the system including an actuated tool and a driver. The articulated tool has a proximal tool interface and a distal portion configured for engaging the tissue. An actuatable feature is disposed along the distal portion and is operatively coupled with the tool interface. The driver is configured to drive the tool, and also has a driver interface. The sterile structure comprises a sterile housing having a receptacle configured to fittingly receive the driver. The sterile housing includes a sterile junction having a sterile barrier extendable between the tool interface and the driver interface so that driver can drive the actuatable feature through the sterile housing when the tool interface is coupled with the driver interface. The sterile housing also has an outer surface and maintains sterile separation between the sterile field adjacent the outer surface and the driver within the sterile housing when the robotic surgical system is configured for use.

In another aspect, the invention provides a catheter for use with a robotic driver system for treating a patient. The catheter comprises an elongate flexible catheter body extending from a proximal catheter housing to an articulated distal portion along a catheter axis. A rotational bearing couples the catheter body to the housing to accommodate manual rotation of the catheter about the axis and relative to the housing during use. A rotational sensor is coupled to the catheter so as to generate catheter rotation state signals during manual rotation of the catheter. The rotation signals will often be sent to the processor so as to induce desired corresponding movements.

In another aspect, the invention provides a sterile interface for use in a catheter system for treating a patient disposed in a sterile field. The system comprises an elongate flexible catheter body having a proximal catheter interface and a distal portion with an axis therebetween. A fluid-actuated feature is disposed along the distal portion and a lumen system provides fluid communication between the fluid-actuated feature and a plurality of catheter fluid receptacles of the catheter interface. A driver assembly has a fluid supply and a driver interface with a plurality of driver fluid receptacles. The sterile interface comprises a sterile junction having a sterile barrier body with a first surface and a second surface opposed to the first surface. A plurality of tubular bodies traverses the sterile body, the tubular bodies having lumenal axes extending between first ends adjacent the first surface and second ends adjacent the second surface. The tubular bodies are supported by the sterile barrier body i) with the axes aligned to facilitate detachably sealed fluid communication between the fluid supply and the fluid-actuated feature, and ii) such that the axes can float sufficiently to accommodate a tolerance of the fluid receptacles.

In another aspect, the invention provides a catheter system for treating a patient. A support surface optionally extends primarily horizontally near the patient, and the system comprises an elongate flexible catheter body having a proximal catheter interface and a distal portion with an axis therebetween. An actuatable feature is disposed along the distal portion and is operatively coupled with the catheter interface. A driver assembly has a power supply, a driver interface releasably couplable with the catheter interface, and a bottom surface or other support feature. The power supply is operatively coupled with the driver interface such that the power supply is drivingly coupled with the actuatable feature when the catheter interface is coupled with the driver interface. The driver is supported relative to the support feature so that when the system is configured for use (optionally with the bottom surface resting on the support surface) with the distal portion of the catheter body in the patient, the catheter interface is oriented primarily downward toward the driver interface. While the power supply may comprise an electrical power supply, in preferred embodiments the power supply will comprise a pressurized fluid source such as a canister containing a gas/liquid mixture.

In yet another aspect, the invention provides a catheter system for treating a patient. The system comprises an elongate flexible catheter body having a proximal catheter interface and a distal portion with an axis therebetween. An actuatable feature is disposed along the distal portion and is operatively coupled with the catheter interface. A driver assembly has a fluid supply, a support feature, and a driver interface releasably couplable with the catheter interface. The fluid supply comprises a receptacle for a pressurized container having a mixture of gas and liquid and configured so that the liquid vaporizes to the gas so as to power movement of the actuatable feature. The receptacle is supported relative to the support feature so that the gas is selectively transmitted out of the container, and un-vaporized liquid remains in the container when the driver assembly is configured for use with the distal portion of the catheter body disposed in the patient.

In another aspect, the invention provides a catheter system for treating a patient. The system comprises an elongate flexible catheter body having a proximal catheter interface and a distal portion with a catheter axis therebetween. A driver assembly has a stand and a driver interface releasably couplable with the catheter interface so as to provide powered movement of the distal portion of the catheter body in the patient. The driver assembly comprises a manual linear motion stage and a support feature, the driver assembly being supported by the support feature relative to the patient. The manual linear motion stage is manually movable along a linear motion axis extending along the catheter axis as to effect movement of the driver interface relative to the support feature during use. Optionally, a sensor can be coupled to the linear motion stage so as to generate signals in response to an axial position of the driver interface relative to the support feature.

In another aspect, the invention provides a fluidically driven tool for use in a robotic surgical system. The system includes a driver having a fluid source and a plurality of fluid drive channels extending toward a driver interface along a plurality of axes. The tool comprises a tool having a distal articulated portion and a proximal catheter interface. The catheter interface comprises an interface housing having an interface wall and a back wall with a plurality of apertures extending through the interface wall. A plurality of coupler bodies is captured between the walls and can slide laterally relative to the axes so that they can be aligned with tubular bodies extending along the channels. A plurality of flexible tubes couples the coupler bodies with the articulated portion.

In another aspect, the invention provides a method for preparing a robotic surgical system for treatment of a patient. The method comprises providing a driver having a plurality of fluid drive channels disposed in a driver housing. The driver can have a driver interface, and may be encased in a sterile housing so that a sterile barrier of the sterile housing extends over the driver interface. A tool interface of a robotic tool can be coupled to the driver interface so that drive fluid from the drive fluid channels can be transmitted through a sterile junction to articulate the robotic tool, the sterile junction comprising the sterile barrier and fluidic coupling components. Advantageously, the sterile barrier can separate an outer surface of the housing from a sterile field that encompasses an access site into the patient.

In another aspect, the invention provides a method for treating a patient. The method comprises manually advancing a robotic catheter from an access site into a patient toward a treatment site. An interface of the robotic catheter can be mounted onto a driver interface of a robotic driver. A user can manually move the driver and the catheter together along a catheter insertion axis so that a target tissue of the patient is within a robotic range of motion of the robotic catheter. The catheter can robotically articulate the catheter within the patient so as to diagnose or treat the tissue.

In another aspect, the invention provides a method for configuring a system for treating a patient. The method comprises coupling a treatment tool with a fluid supply, the tool having a proximal interface and a distal portion with an axis therebetween. The proximal interface is coupled with the fluid supply. The fluid supply is supported for use of the distal portion within the patient, and the fluid supply comprises a pressurized container having a mixture of gas and liquid. The fluid supply is supported so that the liquid vaporizes to the gas and so that the gas is selectively transmitted upward and out of the container toward the distal portion and unvaporized liquid remains in the container.

The above aspects can optionally be combined, as can be understood with the descriptions and drawings provided herein. Relatedly, a number of independent features may be combined with some or all of the aspects provided above. For example, the tools described herein will often comprise an elongate flexible catheter body, and any actuatable feature may optionally comprise an articulatable portion of the catheter body. Preferably, fluid from a fluid supply of the drivers provided herein may fluidically articulate the articulatable portion of the catheter body with the fluid often passing through a sterile junction having a sterile barrier, the sterile barrier forming a component of a sterile housing. Advantageously, the sterile barrier can extend circumferentially around fluid passages through the sterile junction, cooperating with the other elements of the sterile housing to encase and isolate a driver from a sterile field in which the catheter body will be used. The articulatable portion of the catheter will often comprises an articulation balloon array.

As examples of additional independent features that can be included, the fluid supply (which can function as, and may be referred to as, a power supply) may optionally comprise a disposable cartridge containing a pressurized mixture of gas and liquid. The driver will preferable include a plurality of valves and a processor configured to direct the gas from the cartridge along a plurality of fluid channels toward the driver interface. The valves and the processor can be contained in a driver housing, and the outer surface of the driver housing can optionally be configured to be cleaned (such as by being wiped clean), between use on different patients, while the valves and processor remain therein. Alternatively, the driver housing may comprise materials suitable for the driver to withstand gas sterilization, liquid sterilization, radiation sterilization, plasma sterilization, or the like. In contrast to the articulating linkages of manipulators used to induce movement of most known robotic tools, the driver housing will often be unarticulated during use, and/or no motion of the tool may be imparted by movement of a solid structure extending between the driver and the tool. Hence, some of the fluidic drivers provided herein may be described as brick drivers or block drivers. The drive fluid may optionally comprise a gas such as nitrous oxide or carbon dioxide and the canister of the fluid source may optionally have a frangible seal, but in contrast to the fluid systems of cryogenic therapeutic systems, the receptacle will often be oriented, during use, to receive the canister with the frangible seal above the liquid so that un-vaporized liquid primarily remains within the canister and the fluid transmitted from the canister toward the driver interface at least primarily comprises (and ideally essentially entirely consists of) the gas.

Preferably, the sterile housing comprises a semi-rigid or rigid polymer shell having inner features that fittingly receive corresponding outer features of the driver so as to inhibit movement of the driver within the sterile housing. Some sterile housings may comprise flexible membranes. The sterile housing can include a first housing portion with a first housing latch portion and a second housing portion with a second housing latch portion configured to releasable latch with the first latch portion so as to affix the housing portions together with the driver therein, providing a quick-disconnect assembly that can be prepared in or adjacent a treatment room. The second housing portion will often comprise the sterile barrier of a sterile junction, which may be located primarily above the first housing portion when the system is oriented for use, so that the interface of the catheter or other tool is moved down into engagement with the corresponding interface of the driver. In other embodiments, the sterile barrier may extend along a lateral side of the driver (so that the catheter moves laterally relative to the catheter axis into engagement with the driver), or along a proximal or distal side of the driver (so that the catheter moves axially into engagement with the driver). Regardless, a tool latch can releasably affix the tool to the driver through the sterile junction, preferably with a tool latch sensor transmitting signals in response to a state of the tool latch. The processor of the system can be configured to inhibit directing fluid pressure from the fluid source to the driver interface when the latch sensor signals indicate the tool is not safely latched to the driver.

Optionally, the tool comprises an elongate body having an axis extending between the proximal interface and the distal portion. A stand can support the sterile housing so that the housing moves along the axis. In systems that may be particularly beneficial, the system may include an input configured for receiving a movement command and a processor coupling the input with the fluid source so that fluid from the fluid source induces movement of the tool per the movement command. An axial position sensor can be coupled with the stand and can be configured to transmit axial position signals to the processor in response to an axial position of the sterile housing relative to the stand. This may facilitate accurately controlled movement of the elongate body with a patient as the body moves axially into or out of the patient. The axial position sensor (or another sensor of the driver assembly) may also provide signals indicating the driver has been mounted to the stand. Hence, the processor of the system may change in mode in response to detection of driver/stand engagement or disengagement, for example to allow or inhibit articulations. Exemplary sensors which may provide both axial position and engagement signals include force sensitive resistors (FSR's), optoelectronic sensors, and the like.

Sealing of the fluid channels through a removable and replaceable sterile junction of the tool/driver interface may be facilitated by interface sealing bodies that can accommodate lateral and/or orientational displacement. For example, O-rings or other compliant structures having resilient sealing surfaces may be disposed adjacent the ends of tubular bodies of the sterile barrier. This can facilitate sealing with the driver fluid receptacles of the driver interface and the catheter fluid receptacles of the catheter interface. Optionally, to maintain structural integrity of the sterile barrier and accommodate lateral movement relative to the fluid channel axes, a first feature protrudes radially from each of the tubular bodies adjacent the first surface of the sterile barrier body, and a second feature protrudes radially from each of the tubular bodies adjacent the second surface of the sterile barrier body opposite the first surface. The protruding features may comprise split rings, flanges, or the like, and the tubular bodies can have outer profiles between the protruding features such that they can extend through apertures in the sterile barrier body. The apertures can, in fact, be larger than the profiles and the sterile barrier body can be captured between the first and second features so that the tubular bodies can float laterally and/or orientationally throughout a lateral tolerance range, a diametrical tolerance range, or the like. Preferably, the structures of the sterile junction are configured so that the tubular bodies are supported relative to the sterile barrier body axes of the fluid passages can tilt throughout an angle of less than 5 degrees when mounting the catheter to the driver.

Preferably, quick-disconnect latch system preferably holds the tool operatively coupled with the driver when the fluid channels are pressurized, and also facilitates rapid removal and replacement of the tool when desired. Some advantageous tool latch arrangements may include a tension member extending rotatably through the sterile barrier body, the latch tension member comprising a first latch element adjacent the first surface. For example, the first latch element may optionally comprise one or more radially protruding feature such as two opposed protrusions near the end of the tension member in a “T” shaped arrangement. The first latch element can be configured to rotatably engage a driver latch feature of the driver so as to affix the sterile junction to the driver, with the engagement preferably comprising a shelf of a receptacle that provides a cam-and-follower engagement that pulls the sterile junction toward the driver when the T rotates. The latch tension member may also include a second latch element adjacent the second surface (such as one or more protrusions, ideally in a T arrangement), the second latch element configured to be rotatably engaged by a rotating element of the catheter interface so as to affix the sterile junction to the catheter, with the rotating element optionally comprising a shelf of a rotatable receptacle. Hence, at least one (and ideally both) of the latch elements and associated features comprises a cam-and-follower arrangement so that the latch tension member is held in tension when the latch is fully closed. A latch sensor can optionally generate signals indicating the catheter interface is safely latched to the driver.

### BRIEF DESCRIPTION OF THE DRAWINGS

FIG. 1 illustrates an interventional cardiologist performing a structural heart procedure with a robotic catheter system having a fluidic catheter driver slidably supported by a stand.

FIG. 2 is a simplified schematic illustration of components of a helical balloon assembly, showing how an extruded multi-lumen shaft can provide fluid to laterally aligned subsets of balloons within an articulation balloon array of a catheter.

FIGS. 3A-3C schematically illustrate helical balloon assemblies supported by flat springs and embedded in an elastomeric polymer matrix, and show how selective inflation of subsets of the balloons can elongate and laterally articulate the assemblies.

FIG. 4 is a perspective view of a robotic catheter system in which a catheter is removably mounted on a driver assembly, and in which the driver assembly includes a driver encased in a sterile housing and supported by a stand.

FIGS. 5A-5C are an exploded side view, an exploded upper perspective view, and an exploded lower perspective view, respectively, of the robotic catheter system of FIG. 4 showing how a fluidic driver is contained in the sterile housing with a sterile junction having a sterile barrier of the housing disposed in the catheter/driver interface.

FIGS. 6A and 6B are an upper perspective view and a lower perspective view, respectively, of the driver of FIGS. 5A-5C.

FIGS. 7A and 7B are an exploded upper perspective view and an exploded lower perspective view, respectively, of the driver of FIGS. 5A-5C, showing how manifold and processor components of the driver are contained in a housing of the driver.

FIGS. 8A-1-8C are a section view of a proximal catheter housing and associated interface structures, a sterile interface structure, and the driver and associated interface structures, respectively, showing how sterile isolation is provided while allowing drive fluid to flow between the driver and catheter, and also showing how quick-disconnect latch structures facilitate removal and replacement of disposable catheters with the reusable driver.

FIGS. 8D and 8E are schematic perspective detail views of the catheter/driver latch showing coupling of the catheter to the sterile junction and coupling of the sterile junction to the driver, respectively.

FIGS. 8F-8H are schematic perspective detail views of the sealed fluid communication provided between the channels of the driver and the channels of the catheter through the tubular bodies of the sterile junction, and also showing how the tubular bodies are mounted to the sterile barrier body so as to allow the axes of the tubular bodies to float.

FIGS. 8I-8N are exploded detail views showing electrical conductors included in the sterile junction, and how electrical contact is made between circuitry of the catheter and circuitry of the driver when the catheter is mounted to the driver.

FIGS. 9A and 9B are perspective views of the robotic catheter system showing manual sliding of the driver along the catheter axis.

FIGS. 9C-9F are perspective views of drive assemblies having alternative stands with thread systems to allow the user to manually position the sterile housing, driver, and catheter along the catheter axis.

FIG. 9G is a perspective view of an alternative catheter having a manually rotatable catheter body, with a cutaway showing a rotation sensor for transmitting signals to the data processor of the driver in response to an orientation of the catheter body about the catheter axis.

FIG. 9H is a perspective view of driver assemblies having a clamp for releasably axially and rotationally affixing a guidewire relative to the stand.

FIGS. 91-9K are a perspective view, cross-sectional side view, and a detail cross-sectional view of an alternative stand structure having a manually driven thread system with a handle that can be twisted to manually position the driver and catheter in one mode, and that can accommodate manual sliding of the driver relative to the stand supports structure in another mode.

FIGS. 10A-10E illustrate a series of steps that can be used in a method of preparing for and performing an interventional procedure using the devices and systems provided herein.

### DETAILED DESCRIPTION OF THE INVENTION

The present invention generally provides fluid control devices, systems, and methods that are particularly useful for articulating catheters and other elongate flexible structures. The structures described herein are particularly well suited for catheter-based therapies, including for cardiovascular procedures such as those in the growing field of structural heart repair, as well as in the broader field of interventional cardiology. Alternative applications may include use in steerable supports of image acquisition devices such as for trans-esophageal echocardiography (TEE), intra-coronary echocardiography (ICE), and other ultrasound techniques, endoscopy, and the like. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with multiple degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The robotic systems described herein will often include an input device, a driver, and an articulated catheter or other robotic tool. The user will typically input commands into the input device, which will generate and transmit corresponding input command signals. The driver will generally provide both power for and articulation movement control over the tool. Hence, somewhat analogous to a motor driver, the driver structures described herein will receive the input command signals from the input device and will output drive signals to the tool so as to effect robotic movement of an articulated feature of the tool (such as movement of one or more laterally deflectable segments of a catheter in multiple degrees of freedom). The drive signals may comprise fluidic commands, such as pressurized pneumatic or hydraulic flows transmitted from the driver to the tool along a plurality of fluid channels. Optionally, the drive signals may comprise electromagnetic, optical, or other signals, preferably (although not necessarily) in combination with fluidic drive signals. Unlike many robotic systems, the robotic tool will often (though not always) have a passively flexible portion between the articulated feature (typically disposed along a distal portion of a catheter or other tool) and the driver (typically coupled to a proximal end of the catheter or tool). The system will be driven while sufficient environmental forces are imposed against the tool to impose one or more bend along this passive proximal portion, the system often being configured for use with the bend(s) resiliently deflecting an axis of the catheter or other tool by 10 degrees or more, more than 20 degrees, or even more than 45 degrees.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transverse to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the curved outer surface of the cylinder between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

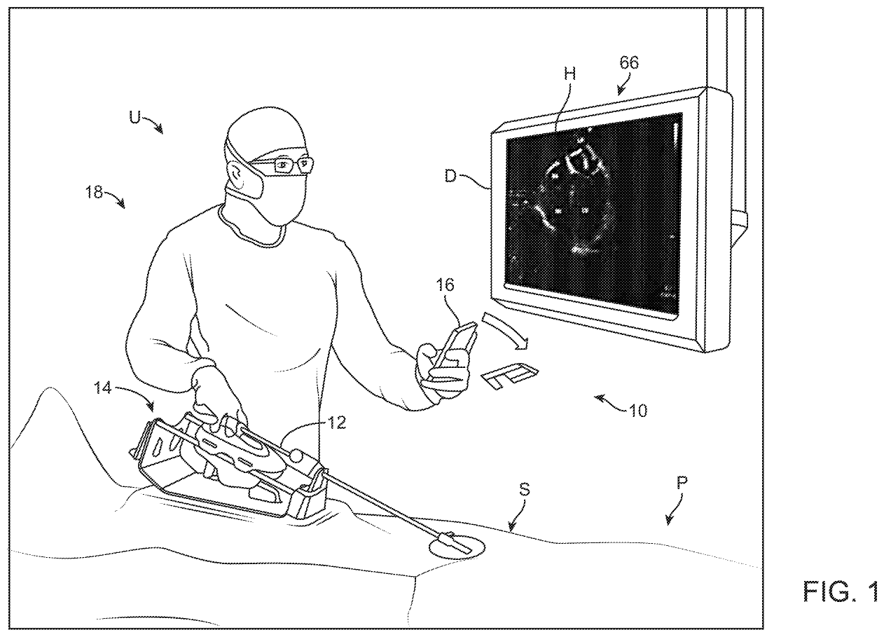

Referring first to FIG. 1, a system user U, such as an interventional cardiologist, uses a robotic catheter system 10 to perform a procedure in a heart H of a patient P. System 10 generally includes an articulated catheter 12, a driver assembly 14, and an input device 16. User U controls the position and orientation of a therapeutic or diagnostic tool mounted on a distal end of catheter 12 by entering movement commands into input 16, and optionally by sliding the catheter relative to a stand of the driver assembly, while viewing a distal end of the catheter and the surrounding tissue in a display D. As will be described below, user U may alternatively manually rotate the catheter body about its axis in some embodiments.

During use, catheter 12 extends distally from driver system 14 through a vascular access site S, optionally (though not necessarily) using an introducer sheath. A sterile field 18 encompasses access site S, catheter 12, and some or all of an outer surface of driver assembly 14. Driver assembly 14 will generally include components that power automated movement of the distal end of catheter 12 within patient P, with at least a portion of the power often being transmitted along the catheter body as a hydraulic or pneumatic fluid flow. To facilitate movement of a catheter-mounted therapeutic tool per the commands of user U, system 10 will typically include data processing circuitry, often including a processor within the driver assembly. Regarding that processor and the other data processing components of system 10, a wide variety of data processing architectures may be employed. The processor, associated pressure and/or position sensors of the driver assembly, and data input device 16, optionally together with any additional general purpose or proprietary computing device (such as a desktop PC, notebook PC, tablet, server, remote computing or interface device, or the like) will generally include a combination of data processing hardware and software, with the hardware including an input, an output (such as a sound generator, indicator lights, printer, and/or an image display), and one or more processor board(s). These components are included in a processor system capable of performing the transformations, kinematic analysis, and matrix processing functionality associated with generating the valve commands, along with the appropriate connectors, conductors, wireless telemetry, and the like. The processing capabilities may be centralized in a single processor board, or may be distributed among various components so that smaller volumes of higher-level data can be transmitted. The processor(s) will often include one or more memory or storage media, and the functionality used to perform the methods described herein will often include software or firmware embodied therein. The software will typically comprise machine-readable programming code or instructions embodied in non-volatile media and may be arranged in a wide variety of alternative code architectures, varying from a single monolithic code running on a single processor to a large number of specialized subroutines, classes, or objects being run in parallel on a number of separate processor sub-units.

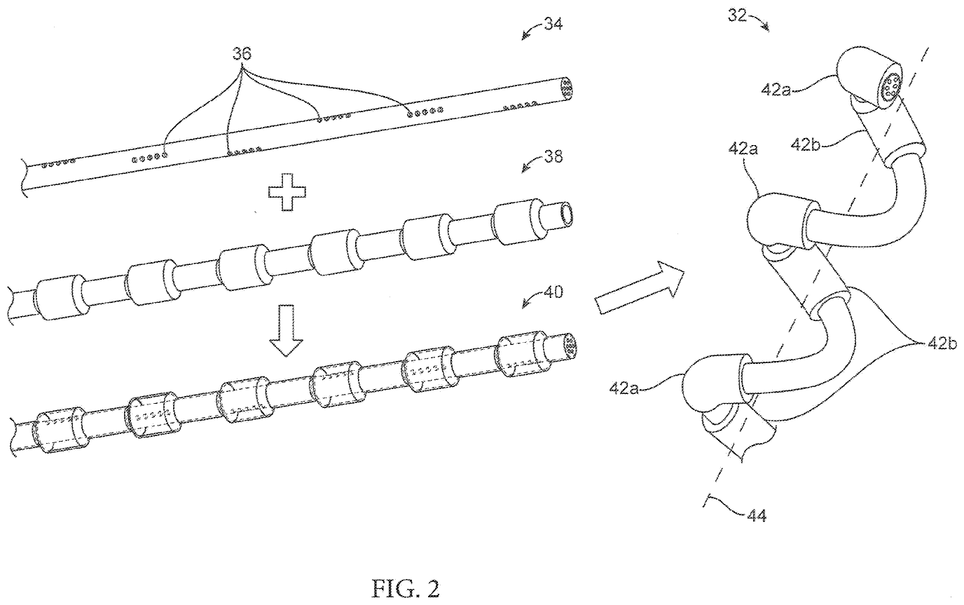

Referring now to FIG. 2, the components of, and fabrication method for production of, an exemplary balloon array assembly (sometimes referred to herein as a balloon string 32) can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using a compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The ports can be formed by laser drilling or mechanical skiving of the multi-lumen shaft with a mandrel in the lumens. Each lumen of the shaft may be associated with between 3 and 50 balloons, typically from about 5 to about 30 balloons. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

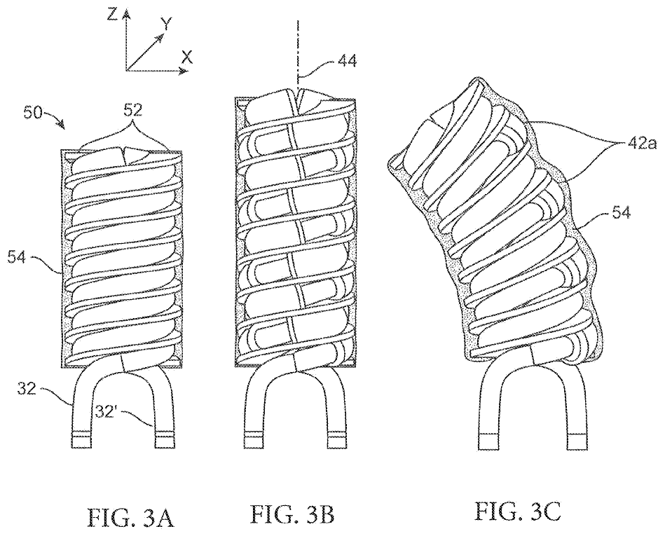

Referring now to FIGS. 3A, 3B, and 3C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 3A and 3B, inflation of subsets of the balloons surrounding the axis of segment 50 can induce axial elongation of the segment. As can be understood with reference to FIGS. 3A and 3C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 or 7 lumens), so that the total balloon array may include a series of independently articulatable segments (each having 3 or 4 dedicated lumens of one of the multi-lumen shafts, for example). Optionally, from 2 to 4 modular, axially sequential segments may each have an associated tri-lumen shaft, with the tri-lumen shaft extending axially in a loose helical coil through the lumen of any proximal segments to accommodate bending and elongation. Multi-lumen shafts for driving of distal segments may alternatively wind proximally around an outer surface of a proximal segment, or may be wound parallel and next to the multi-lumen shaft/balloon tube assemblies of the balloon array of the proximal segment(s).

Referring still to FIGS. 3A, 3B, and 3C, articulated segment 50 optionally includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Alternatively (or in addition to a relatively soft matrix), a thin layer of relatively high-strength elastomer can be applied to the assembly (prior to, after, or instead of the soft matrix), optionally while the balloons are in an at least partially inflated state. Advantageously, matrix 54 can help maintain overall alignment of the balloon array and springs within the segment despite segment articulation and bending of the segment by environmental forces. Regardless of whether or not a matrix is included, an inner sheath may extend along the inner surface of the helical assembly, and an outer sheath may extend along an outer surface of the assembly, with the inner and/or outer sheaths optionally comprising a polymer reinforced with wire or a high-strength fiber in a coiled, braid, or other circumferential configuration to provide hoop strength while accommodating lateral bending (and preferably axial elongation as well). The inner and outer sheaths may be sealed together distal of the balloon assembly, forming an annular chamber with the balloon array disposed therein. A passage may extend proximally from the annular space around the balloons to the proximal end of the catheter to safely vent any escaping inflation media, or a vacuum may be drawn in the annular space and monitored electronically with a pressure sensor to inhibit inflation flow if the vacuum deteriorates.

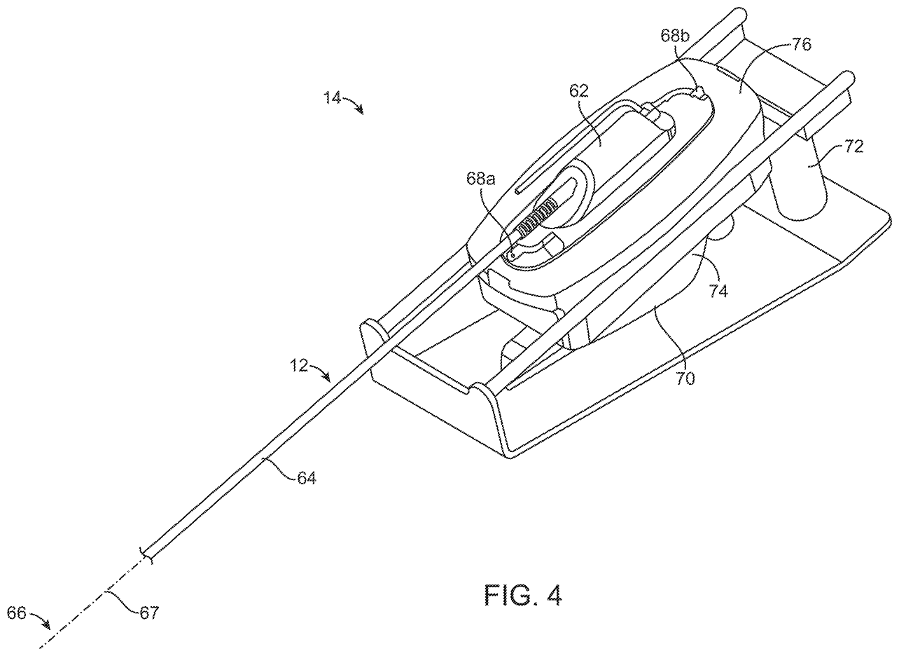

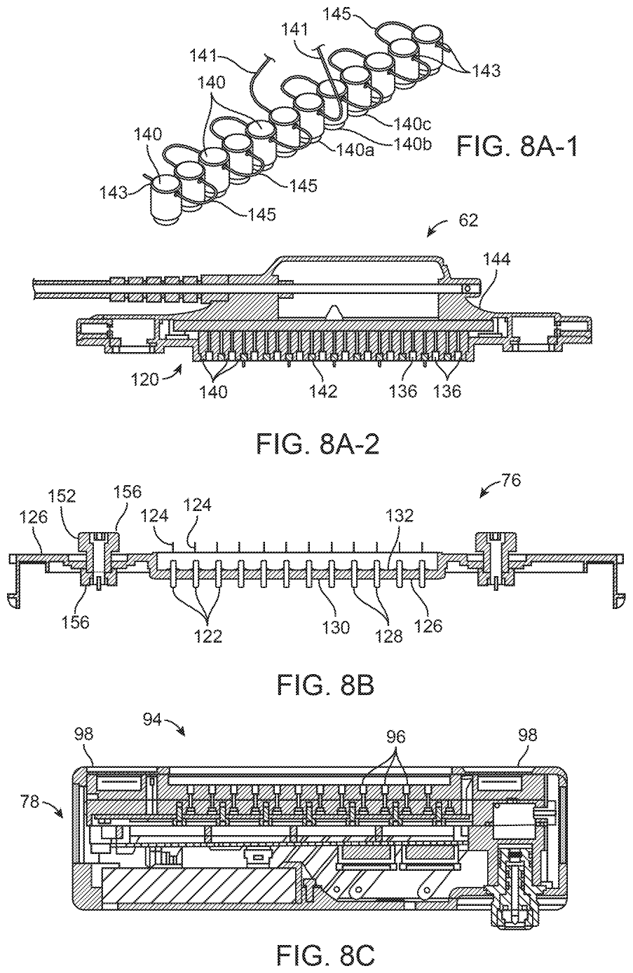

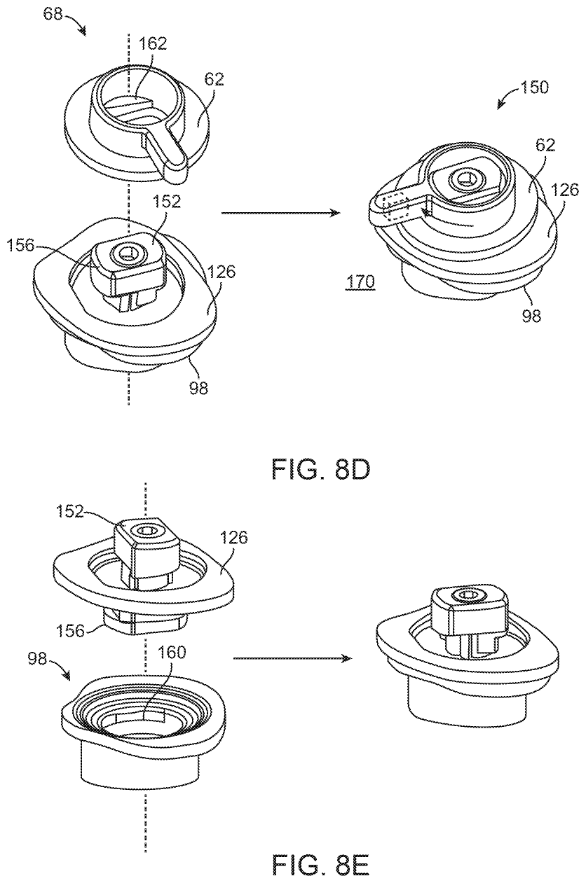

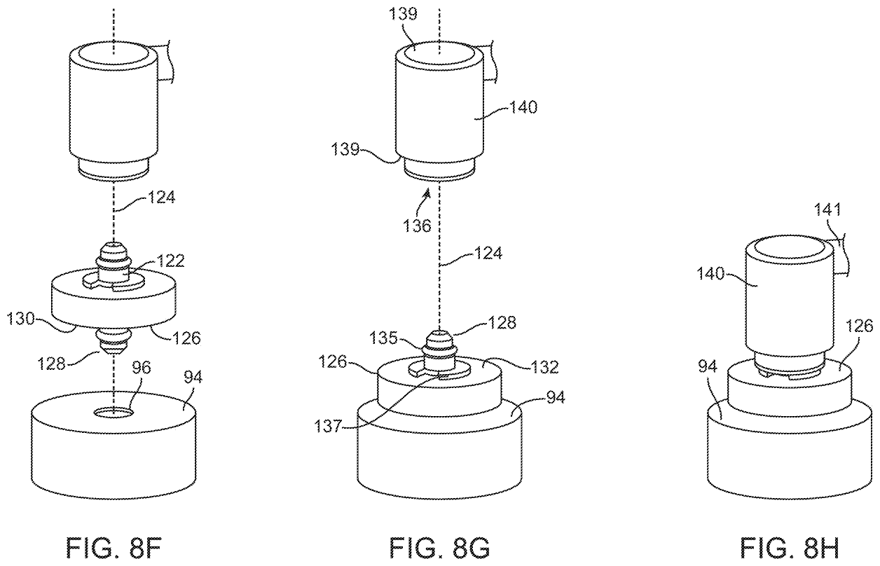

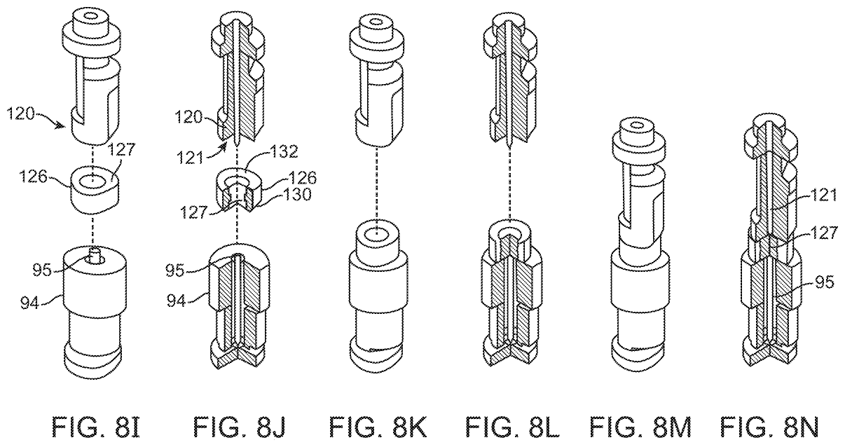

Referring now to FIG. 4, a proximal housing 62 of catheter 12 and the primary components of driver assembly 14 can be seen in more detail. Catheter 12 generally includes a catheter body 64 that extends from proximal housing 62 to an articulated distal portion 66 (see FIG. 1) along an axis 67, with the articulated distal portion preferably comprising a balloon array and the associated structures described above. Proximal housing 62 also contains first and second rotating latch receptacles 68a, 68b which allow a quick-disconnect removal and replacement of the catheter. The components of driver assembly 14 visible in FIG. 4 include a sterile housing 70 and a stand 72, with the stand supporting the sterile housing so that the sterile housing (and components of the driver assembly therein, including the driver) and catheter 12 can move axially along axis 67. Sterile housing generally includes a lower housing 74 and a sterile junction 76 having a sterile barrier body 126 (see FIG. 8B). Sterile junction 76 releasably latches to lower housing 74 and the sterile barrier body extends between catheter 12 and the driver contained within the sterile housing, the sterile barrier body surrounding fluid passage components of the sterile junction. Along with components that allow articulation fluid flow to pass through the sterile fluidic junction, the sterile junction may also include one or more electrical connectors or contacts to facilitate data and/or electrical power transmission between the catheter and driver, such as for articulation feedback sensing, manual articulations sensing, or the like. The sterile housing 70 will often comprise a polymer such as an ABS plastic, a polycarbonate, acetal, polystyrene, polypropylene, or the like, and may be injection molded, blow molded, thermoformed, 3-D printed, or formed using still other techniques. Polymer sterile housings may be disposable after use on a single patient, may be sterilizable for use with a limited number of patients, or may be sterilizable indefinitely; alternative sterile housings may comprise metal for long-term repeated sterile processing. Stand 72 will often comprise a metal, such as a stainless steel, aluminum, or the like for repeated sterilizing and use.

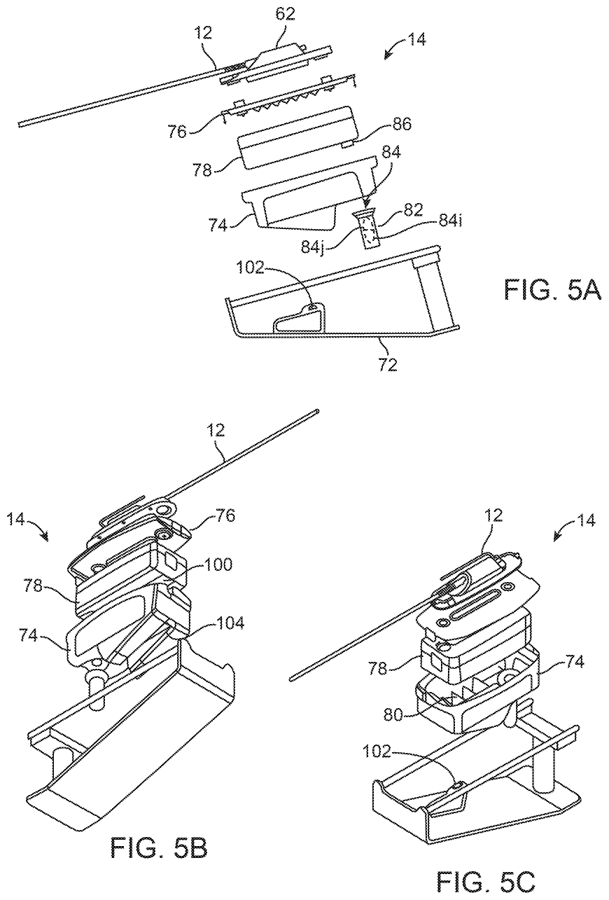

The exploded views of FIGS. 5A-5C illustrate additional components and features of driver assembly 14. In particular, features 80 within lower housing 74 fittingly receive and support a driver 78. A tension member of the catheter latch system extends through a sterile barrier of a sterile junction 76 between catheter housing 62 and driver 78 to keep these structures coupled together when significant fluid pressure is transmitted between the fluid channels of the catheter and driver. Driver 78 is also sufficiently contained within sterile housing 70 to maintain the integrity of the sterile field surrounding driver assembly 14 even if the outer surface of the driver has, for example, been cleaned using a wipe-down approach, but has not been fully sterilized sufficiently for exposed use in the sterile field. Driver 78 also includes a receptacle for a gas/liquid fluid source and is oriented so as to at least primarily receive gas. Typically, sterile cover 82 for a disposable nitrous oxide canister 84 threads onto a canister connector 86 of the driver so as to pierce a frangible seal of the canister and allow pressurized gas to flow from an upper portion of the canister into the fluid channels of the driver. More generally, canister 84 contains a gas 84g near the upper portion of the canister, and a liquid 841 in the lower portion of the canister. As the canister is supported relative to the other components of the driver assembly (and eventually relative to the bottom surface of the stand) so that the fluid used to power the balloon array will be taken from adjacent the top of the canister. In contrast to cryogenic applications, the unvaporized liquid will largely remain in the canister and primarily gas will be transmitted toward the valves and on toward the driver interface. A number of alternative pressurized fluid sources could also be used, including bottles or canisters of N2O, CO2, N2, or the like. Regardless, the gas may optionally be used to pressurize a plenum of liquid such as saline or the like, particularly when it would be desirable to use a non-compressible inflation medium in the balloon array.

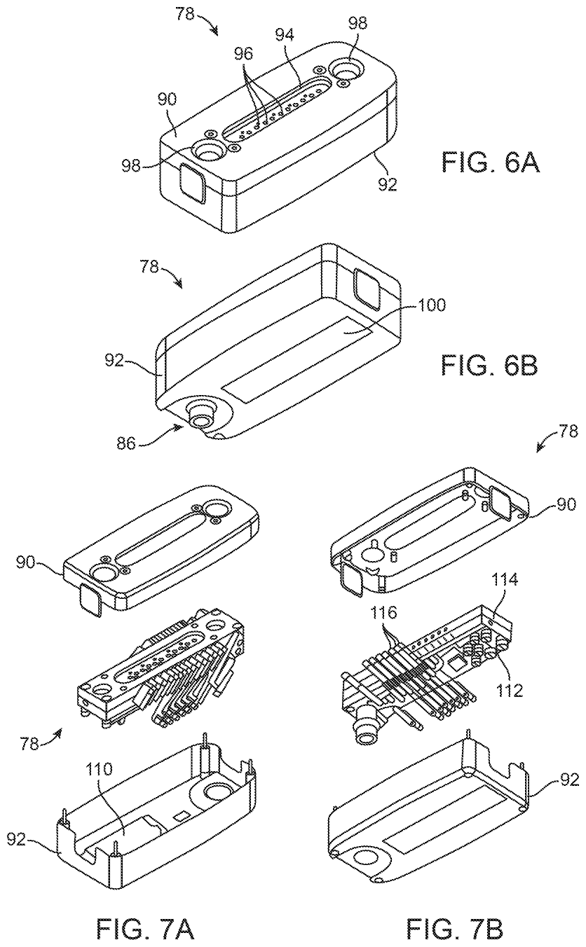

Referring now to FIGS. 6A-7B, additional features of driver 78 can be seen in more detail. A driver housing defines much of the outer surface of the driver, with the driver housing including an upper driver housing 90 and a lower driver housing 92. The driver housing will typically comprise a polymer such as one of the polymers identified above for use in the sterile housing. A driver interface 94 is configured to couple the driver to a catheter during use, with the driver interface having a series of channel openings 96 adjacent one or more quick-disconnect latch receptacle 98, the latch system preferably including a plurality of receptacles or other components arranged with the channel openings therebetween. An axial position sensor 100 couples with a feature of the stand to generate axial position signals indicative of a position of driver along an axis of the catheter, with the exemplary axial sensor comprising a force sensitive resistor (FSR) positioned to be actuated by a protrusion or wheel of stand 72 through a membrane 104 of sterile housing 70 (see FIGS. 4, 5A, 5B).

As can be seen in FIGS. 7A and 7B, along with the components of the driver housing, driver 78 includes fluid control components, data processing components, and a battery 110. The data processing components will typically include one or more printed circuit board (PCB) 112 with circuit components (including integrated circuits) embodying machine readable code configured to generate command signals in response to input from the user. The fluid handling components will include a manifold assembly 114 having an array of valves 116, and the valves will be actuated by the command signals from the PCB 112 so as to direct fluid from the canister receptacle toward the channel openings of the driver interface and also to allow the fluids to be drained from the drive interface. The manifold will often comprise a metal such as a stainless steel, an aluminum, or the like, and the material of the manifold may also define the driver receptacle. The driver and driver housing will often be configured for long-term use, such as to treat dozens or even hundreds of patients, with the outer surface of the driver being wiped clean between patients using standard surgical room antiseptics. Alternatively, the driver housing may be replaced after more limited use (or even a single patient), or the driver may be sealed (such as using one or more removable fixture to block the driver channel openings 96 (FIG. 6A) and canister receptacle 86 (FIG. 6B)) sufficiently for chemical bath or other sterilization techniques.

Referring now to FIGS. 8A-1-8C, additional structures associated with (and relationships between) the interface 94 of driver 78 and receptacle 120 of catheter housing 62 are shown. Associated details of the fluidic coupling between the fluidic components of driver interface 94, catheter receptacle 120 via tubular bodies 122 of sterile junction 76 can be seen in FIGS. 8F-8H. As noted above, fluid channel openings 96 of the driver interface are disposed in an array along an axis, but can be distributed in 2-dimensional patterns in other embodiments. A corresponding array of tubular bodies 122 are included in sterile junction 76, with the tubular bodies and driver channel openings 96 being aligned along parallel axes 124 that are similarly spaced. As can be understood with reference to the detail view of FIG. 8F, tubular bodies 122 are supported along a plate-like region of a sterile barrier body 126 so that driver ends 128 of the tubular bodies extending from a first surface 130 of the sterile barrier body can be advanced together into channel openings 96 of the driver interface 94. The tubular bodies will often comprise a metal (such as stainless steel or aluminum) or a polymer. As can be understood with reference to the detail view of FIG. 8G, opposed ends 128 of the tubular bodies adjacent a second surface 132 of sterile barrier body 126 can similarly be advanced in unison into fluid channel openings 136 of catheter interface 120. Optionally, both ends of the tubular bodies include a compliant surface for sealing against the surrounding fluid channel openings, such as by including O-rings 135, molding or over-molding the tubular bodies with elastomeric materials, or the like. Alternatively, tubular bodies might be associated with the driver interface or the catheter interface, or both, with corresponding receptacles on adjacent sides of the first surface 130 and second surface 132 of the sterile coupler, or any combination of the above.

To accommodate any separation distance or angular mismatch between the fluid channel openings 96, 136 and tubular bodies 122, the sterile barrier body may support the tubular bodies so as to allow them to float within a tolerance range, for example, by over-molding a softer material of the sterile barrier body 126 over a more rigid material of the tubular bodies or the like. Preferably, the tubular bodies extend through oversized apertures through the sterile barrier body 126, with radially protruding split-rings 137 or flanges attached to the tubular bodies adjacent the opposed surfaces 130, 132 capturing the sterile barrier body but allowing the tubular bodies to slide laterally and/or rotate angularly within the apertures. In a somewhat analogous arrangement, channel openings 136 of catheter interface 120 may float laterally by forming each opening in a separate coupler body, often referred to herein as a puck 140. While the preferred coupler bodies are cylindrical, other coupler bodies may have rectangular or other cross sections. The orientation and general position of the catheter channel openings can be maintained by capturing flat surfaces 139 of pucks 140 between a first wall 142 and a second wall 144 of the catheter interface, allowing the pucks to slide laterally within a tolerance range to accommodate spacing of the tubular bodies when the opposed ends extend into the channel openings 96 of the driver interface 94. Apertures through first wall 142 may accommodate the tubular bodies to facilitate coupling, or pucks 140 surrounding openings 136 may extend through the apertures (a protruding portion of the puck being smaller than the aperture to accommodate the axial float tolerance). Note that the ends 122 of the tubular bodies and/or the channel openings 96, 136 may be chamfered to facilitate engagement, and a series of flexible polymer tubes 141 may be bonded or otherwise affixed to the pucks 140, with the tubes extending into the catheter body or otherwise providing fluid communication between the catheter interface and balloon array.

Referring now to FIG. 8A-1, fluid coupling between pucks 140 and multi-lumen inflation fluid shafts 141 is shown without walls 142, 144 or other structures of the proximal catheter housing for clarity. One or more multi-lumen shafts 141 extend from an array of pucks 140 within the housing and distally into the catheter body to provide fluid coupling between the catheter interface 120 and the balloon array. The pucks have apertures extending therethrough, and a first multi-lumen shaft 141 from the catheter body extends through the aperture of a first puck 140a, then through at least one additional aperture of a second puck 140b, preferably to a third puck 140c, and optionally through a total of 4, 5, 6, or more pucks. A second multi-lumen shaft may extend through additional pucks of the puck array, and still additional multi-lumen shafts may be included. The pucks have channel openings 136 and at least one radial port is provided within each puck into an associated lumen of the multi-lumen shaft extending therethrough (similar to ports 36 of FIG. 2), so as to provide fluid communication between the channel opening of a particular puck 140 (such as puck 140a) and a particular lumen of the multi-lumen shaft. The multi-lumen shaft(s) 141 are bonded within the apertures 143, and a length 145 of multi-lumen shaft between adjacent pucks is longer than a separation distance between adjacent apertures 143 so that a bend of the multi-lumen shaft can accommodate relatively movement between the pucks 140. Optionally, the multi-lumen shafts have between 2 and 21 lumen, preferably having 3, 6, 8, 9, or 12 lumens.

Referring now to FIGS. 8A-1-8E, the structure and use of quick-disconnect latches 150 are shown in more detail. Latches 150 releasably affix the driver interface to the catheter interface (with the sterile junction disposed therebetween) using a tension member 152 that extends from receptacle 98 of the driver, through sterile barrier body 126, and into receptacle 68 of catheter housing 62. Tension member 152 is rotatably mounted to sterile barrier body 126, and each end of the tension member has circumferentially opposed protrusions, thereby forming T-shaped member ends 156. As seen best in FIG. 8E, a first T end 156 can be advanced into driver receptacle 98 along an axis of tension member 152 when the T is in a first orientation, while the tubular bodies 120 of the sterile junction are being advanced into engagement with the openings 96 of driver interface 94. Tension member 152 can then be rotated about its axis, bringing a surface of the T 156 into engagement with a shelf 160. T 156 preferably floats somewhat within its receptacle prior to attachment of the catheter. Subsequent attachment of the catheter draws T 156 up into a recess of sterile body 126, which locks it in place to inhibit loosening of the sterile junction if the catheter is removed. Shelf 160 and/or T 156 could alternatively be sloped so that the rotation draws tension member 152 down into receptacle 98, promoting movement of tubular bodies 122 into opening 96. A stop and/or detent can maintain the latched rotational orientation of tension member 152 so as to inhibit movement of the sterile junction away from the driver. Subsequently, receptacles 68 of catheter housing 62 can be moved axially down over the oriented T 156 on the opposed end of tension member 152. Receptacles 68 are rotatably mounted to the adjacent body of the catheter housing, and have shelves 162 that engage the adjacent T surface when the catheter latch receptacles are rotated into a latched orientation, as can be seen in FIG. 8D. Shelves 162 and/or the engaged surfaces of T 156 are sloped so as to urge receptacle 68 down over tension member 152, and so as to pull the T upward within the driver receptacle 98. A sensor 170 of the driver may sense that the rotatable catheter receptacles are in the latched orientation, optionally using a hall-effect sensor or the like. Friction, stops, and/or detents can help maintain the latched configuration of the receptacles and tension member, and the driver processor may inhibit transmission of fluid into the channels of the catheter (and/or may drain any pressure from the channels) if latch 150 is not in a safe, fully latched configuration. The tension members may comprise any of the metals or polymers described above, optionally having a metal core or shaft therein, with a spring to help hold the latch in a fixed configuration. The use of a spring may be particularly beneficial to facilitate latching of the tension member with the catheter housing, and to inhibit falling of the tension member downward after latching of the sterile junction to the base and prior to latching of the catheter housing.

Referring to FIGS. 8A-1-8C, and 8I-8N, along with the fluidic coupling described above, sterile junction 76 may also provide sterile electrical coupling of driver contacts 95 of driver interface 94 with catheter contacts 121 of catheter receptacle 120. Toward that end, electrical conductors 127 may be mounted to sterile barrier body 126, with the conductors preferably comprising a conductive material extending from first surface 130 to second surface 132. Conductors 127 may comprise a metal that is screwed, press fit, adhesively bonded, or the like, into the polymer material of the sterile barrier body so as to have exposed conductive surface on the opposed major surfaces. Driver contacts 95 and 121 may comprise metal structures biased to engage the conductors when the catheter is latched to the driver with the sterile junction therebetween, with the exemplary contacts comprising spring pins mounted in the polymer materials underlying the driver interface and the catheter receptacle. The exploded detail views of FIGS. 8I, 8K, and 8M show the conductive components with a small amount of the surrounding polymer structures and the catheter receptacle, sterile junction, and driver interface separated (in FIG. 8I), with the sterile junction mounted to the driver interface (FIG. 8K), and with the catheter mounted to the driver through the sterile junction (FIG. 8M), respectively. The structure and resilient deflection of the drivers is seen most clearly in the corresponding quarter cross-sections of FIGS. 8J, 8L, and 8N.

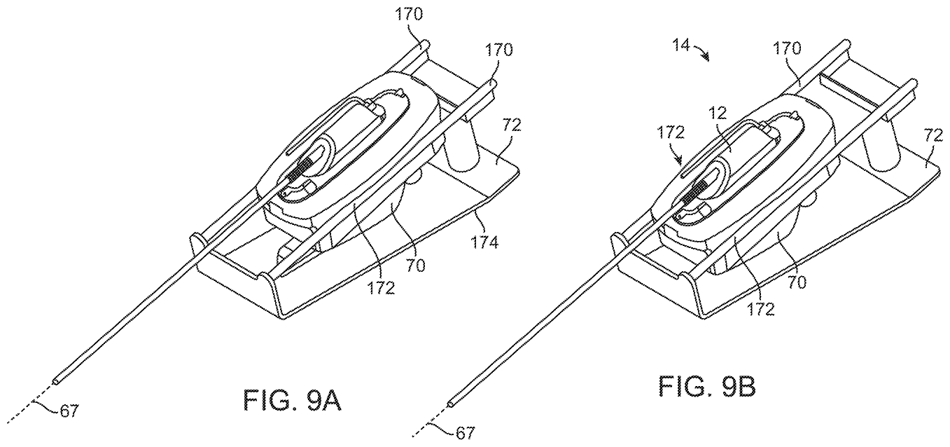

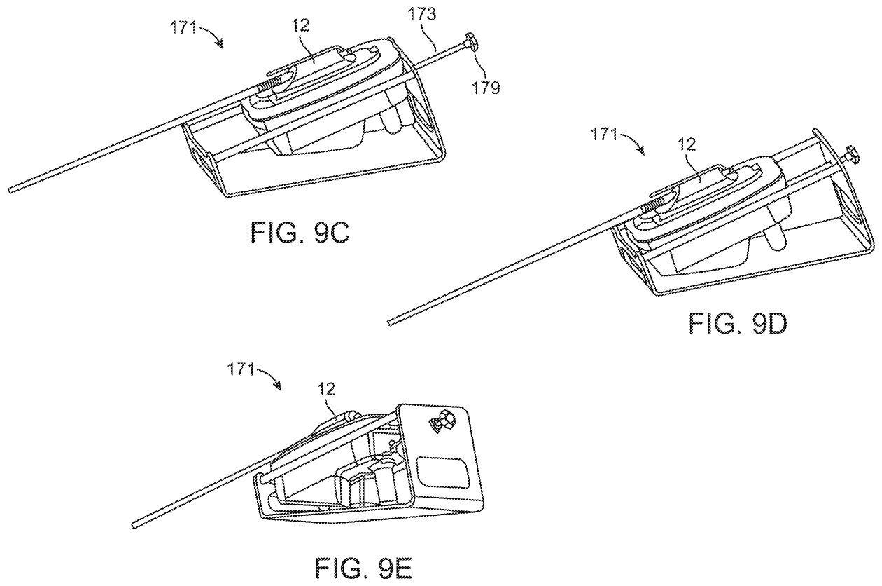

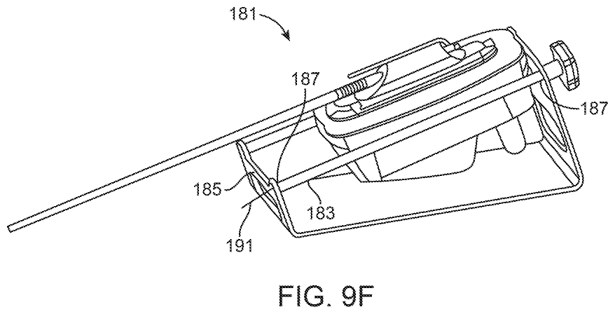

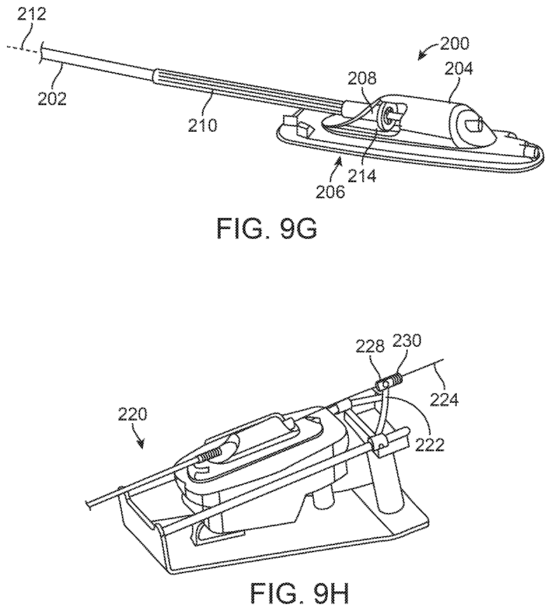

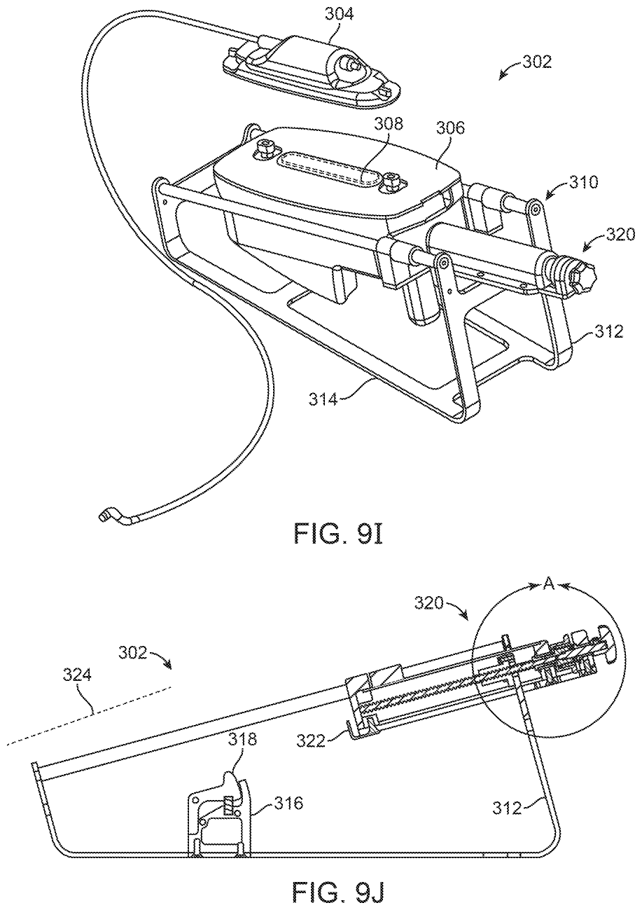

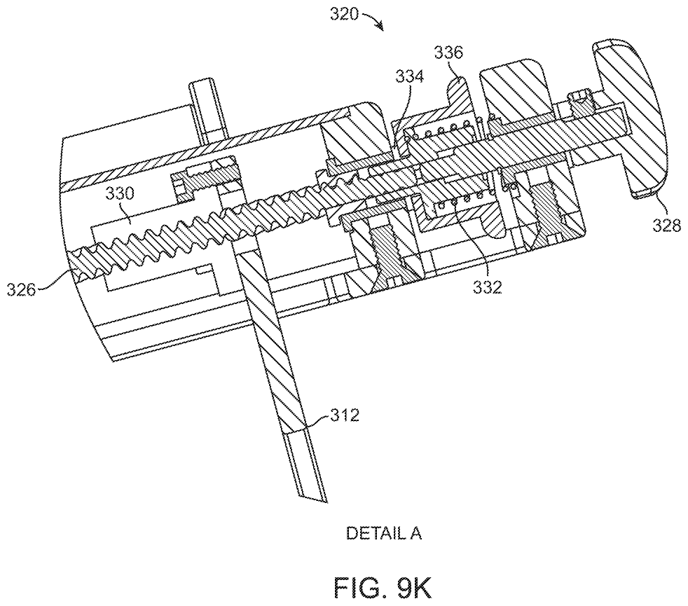

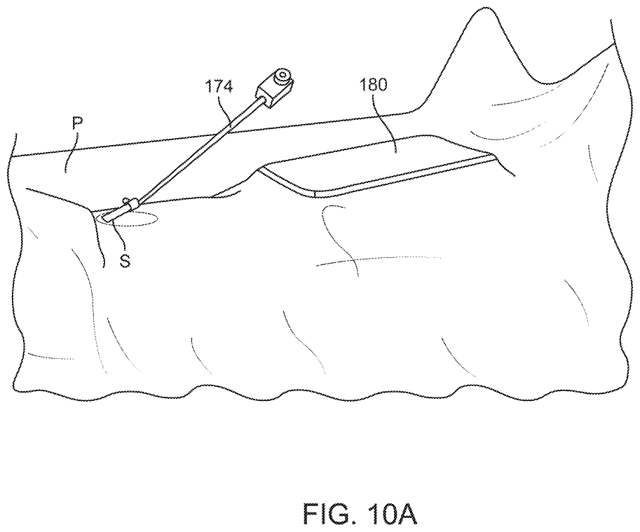

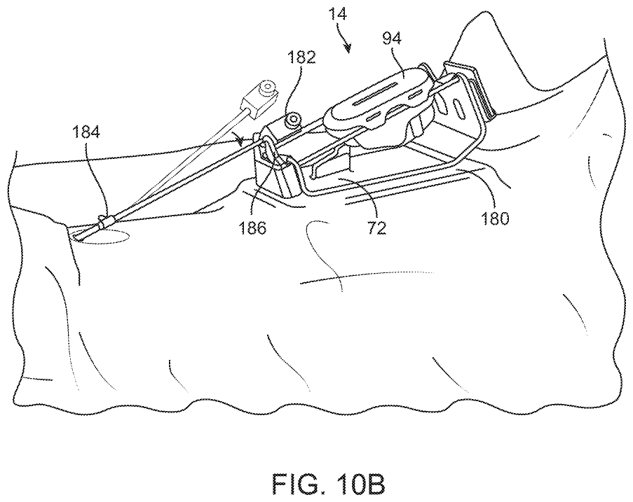

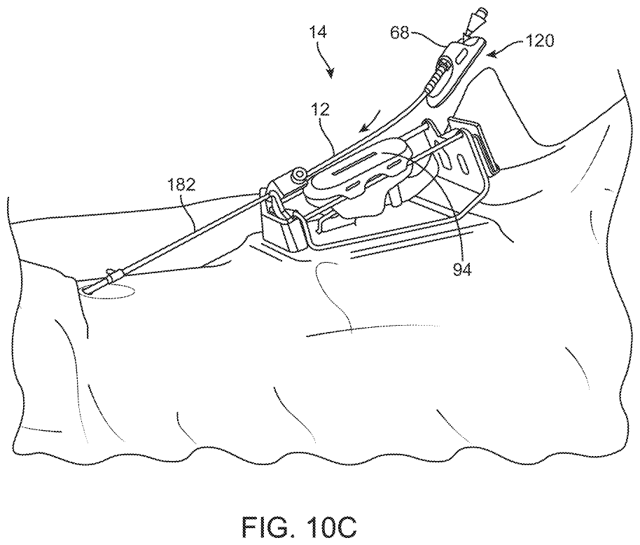

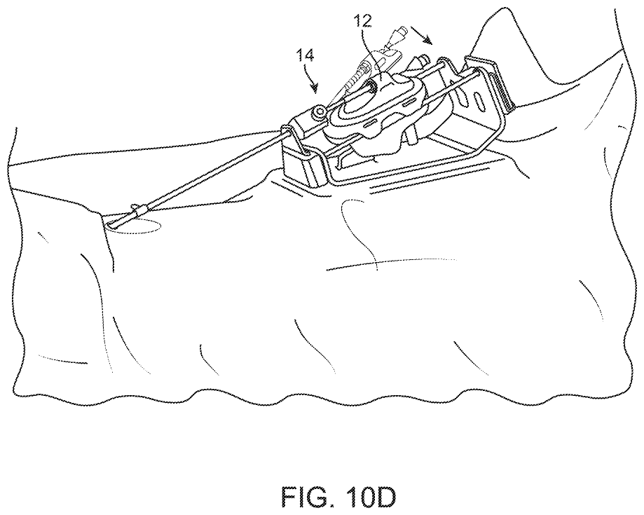

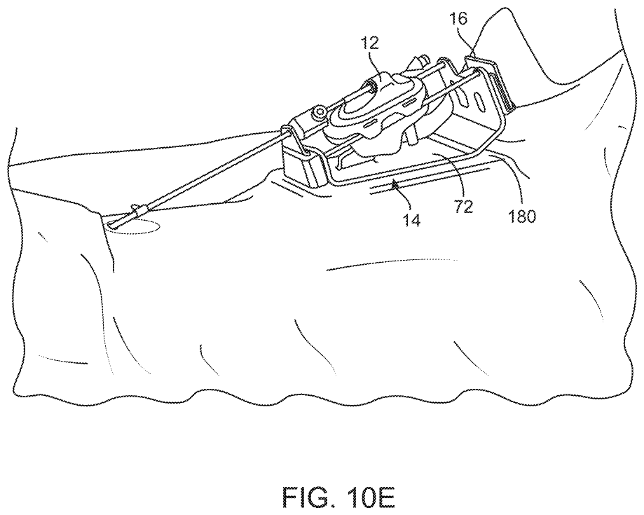

Referring to FIGS. 5A-5C, 9A, and 9B, the structures which allow and sense movement of catheter 12 and driver assembly 14 relative to stand 72 can be understood. Stand 72 includes a pair of rails 170 that extend along catheter axis 67. Sterile housing 70 includes a pair of bearing surfaces 172 that slidingly engage rails 170, the bearing surfaces being formed by locally protruding flanges. Bearing surfaces 172 may optionally be integral with the sterile housing, such as by forming the outer surface of the sterile housing material when the sterile housing is molded or 3-D printed. Alternatively, a low-friction layer or region (such as can be formed by bonding a flexible layer or tape of PTFE or another low friction polymer to the underlying sterile housing) may be provided for use as the bearing surface. Regardless, the system user may move the catheter and driver structures axially relative to stand 72 and the patient along the catheter axis (and hence advancing the catheter distally within or retracting the catheter proximally out of the patient) by manually sliding the shelves 172 along rails 170, so that the shelves and rail act as a simple manual linear motion stage. As the sterile housing 70 moves, protruding roller 102 locally deflects membrane 104 of the sterile housing toward the driver therein, pushing the membrane against sensor 100 of the driver. The sensor transmits signals to the processor of the driver, allowing the driver to calculate revised command signals appropriate for a change in axial position of the catheter within the patient. Note that stand 72 will often be used while a bottom surface 174 of the stand rests on a primarily horizontal support surface. The bearing surfaces 172 of the sterile housing support the sterile housing relative to stand 72, and the internal features of the sterile housing, in turn, help position and support the driver relative to the sterile housing. The stand may optionally be slid along the surface on which the bottom of the stand is resting to effect movement along the axis, typically at a slight angle (for example, from 5 to 25 degrees) relative to catheter axis 67. In other embodiments, an alternative support feature may be used in place of bottom surface 172, for example, with the support feature comprising a clamp configured to attach to a rail extending along a surgical table or other support surface.

As generally described herein, the driver assembly may optionally be configured to facilitate manual positioning of the catheter along the catheter axis, for example, by accommodating axial movement of the sterile housing relative to the stand. This can allow the user to input commands to the robotic catheter system to urge the distal articulated portion of the catheter toward a desired shape before, during, and/or after axial catheter movement, and as the axial position and movement can be sensed by the axial sensors described herein, the axial movement may also be used as an input to the catheter drive system. As described above, the manual movement of the catheter may optionally be induced by sliding the catheter along rails of the stand. Optionally, movement of the catheter (and driver) can be induced by the user manually rotating a handle of a threaded rod coupling the stand to the driver. For example, referring to FIGS. 9A-9C, an alternative driver assembly 171 includes many components related to those described above, but here has a threaded rod 173 axially coupling sterile housing 175 to stand 177. Threaded rod 173 has a handle 179 that can be rotated manually by the user, and a rotatable bearing affixes an end of the threaded rod to the sterile housing 175, while a nut or threaded bearing changes the axial position of the threaded rod, sterile housing, driver, and catheter 12 when the handle is rotated. A simplified threaded driver assembly 181 is shown in FIG. 9F, in which a threaded rod or rail 183 is coupled with a frame 185 of the stand by proximal and distal rotatable bearings 187, allowing a handle 189 to rotate the threaded rod about its axis 191. Axis 191 extends along (preferably being parallel to) the axis of the catheter body, and a threaded surface 193 of sterile housing 195 is configured to engage the threaded surface of threaded rod 183 when the sterile housing rests on the threaded rod and a smooth cylindrical rail 170. This allows the user to drive the catheter axially by manually rotating the handle, and to manually tilt or lift the threaded surface of the sterile housing clear of the threaded rod to move the catheter axially relative to the frame (or the frame relative to the catheter) without rotating the handle. The tilting or lifting of the sterile housing may decouple the axial sensor of the driver from the protrusion of the frame (SEE FIGS. 5B, 5C, and 6B), generating a signal that can be used by the processor, for example, to reset an axial relationship between the catheter and frame.

Referring now to FIG. 9G, a rotatable shaft catheter 200 shares many of the structures of the catheters described above, including a catheter body 202 extending distally from a proximal catheter housing 204 having a catheter receptacle 206 configured for coupling with a driver. Catheter body 202, however, is rotationally attached to housing 204 by a rotational bearing 208 that allows the user to manually rotate the catheter body about the catheter axis. A handle 210 is mounted to the catheter body near bearing 208. The handle is configured to be grasped by the hand of the user and rotated about axis 212. A sensor 214 senses the rotational state of the catheter and transmits catheter rotation signals to the processor of the driver, optionally via conductors of the sterile junction as described above with reference to FIGS. 8I-8N. Sensor 214 may comprise an optical encoder, a potentiometer, or the like. The signals will be suitable for providing real-time feedback on the catheter rotational state to the processor so as to allow the processor to calculate articulation drive signals for the articulated portion of the catheter. Note that a wide variety of alternative rotational or axial sensors may be provided, either sensing positional relationships adjacent the driver, along a length of the catheter assemblies, or the like. In some embodiments, the rotation (or axial offset) may be measured distally of housing 204, such as using an encoder or resistor affixed to a distal portion of a guide catheter surrounding catheter body 202 adjacent the articulated portion, and an optical sensing surface or electrical contact mounted to the catheter body.

Referring now to FIG. 9H, an alternative driver assembly 220 has a guidewire support 222 to axially and/or rotationally affix a guidewire 224 relative to a stand 226. The driver assembly may otherwise share some, most, or all of the features described above regarding each of the driver assemblies. Guidewire support has a lateral opening 228 to receive guidewire 224 laterally (relative to the axis of the guidewire) into jaws of the support. A guidewire rotational knob 230 may be affixed rotationally to the guidewire by a set screw or the like, In methods that avoid the use of a guide catheter such as that shown affixed to a distal clamp of the stand by support 186, a guidewire (such as a super stiff guidewire or extra stiff guidewire) may instead be affixed to guidewire support 222 of the stand proximally of the driver, typically after catheter 12 is loaded retrograde onto the guidewire and has been advanced so that a distal end of the catheter is adjacent the target tissue (and so that the proximal housing of the catheter is distal of the proximal guidewire support or clamp). The stand may include both a distal releasable clamp or support 186 for the guide catheter (as shown above) and a releasable proximal clamp or support 222 for the guidewire 224 proximal of the rails. Both the guide catheter clamp and guidewire clamp may be used together for some procedures, with the guidewire often ending proximally of (or having only a highly flexible distal portion extending into) the articulated portion of the catheter, which will often extend distally of (or be articulated distally of) the distal end of the guide catheter.

Referring now to FIGS. 91-9K, a still further alternative driver assembly 302 includes any of the fluid-driven catheters 304 sterile housings with associated sterile junctions 306, and drivers 308 described herein, along with an alternative stand assembly 310. Stand assembly 310 includes a stand support structure 312 having a bottom surface 314 configured to rest on a roughly horizontal support surface such as a cantilevered over-bed support table, a small table that can be placed on the surgical table over the patient leg, or the like. As may be seen best in FIG. 9J, driver assembly 302 also includes an axial position indicating assembly 316 having a pivotally supported surface 318 that is biased to engage and slide against an axial position sensor of the driver.

As can be seen most clearly in FIGS. 9J and 9K, stand assembly 310 also includes a multi-mode axial drive assembly 320. Drive assembly 320 includes a catch 322 which is configured to axially engage the sterile housing and facilitate driving of the housing (and components of the driver assembly supported thereby) along the catheter axis 324. An axial drive thread 326 is rotationally coupled to a handle 328 and engages a nut 330. Nut 330 is axially affixed to the support structure 322 of the stand, so that rotation of the handle drives the catch and handle along the catheter axis. To inhibit inadvertent rotation of the handle and associated axial sliding of the driver and catheter along the catheter axis in a rotational positioning mode, a spring 332 biases a brake surface 334 against a corresponding surface rotationally affixed to the stand structure 312. The break surface may be defined by a polymer, metal, or the like, and the engagement will typically be sufficient to prevent the catheter from being moved downward along the sloping catheter axis by the weight of the driver, but not sufficient to make intentional manual rotation of the handle and resulting axial positioning of the catheter too onerous when the stand is being used in the threaded positioning mode, such as for accurate manual advancement of the end of the catheter within a chamber of the heart. When faster manual positioning of the driver is desired, such as prior to or during mounting of the catheter to the driver, a collar may be pulled (and optionally latched) in a proximal position by the user, disengaging the brake from the corresponding surface and allowing thread 326 to rotate more freely so that the user can manually slide the driver along the catheter axis without having to rotate handle 328.

Referring now to FIGS. 1 and 10A-10E, a method for preparing robotic system 10 for use can be understood. As seen in FIG. 10A, a horizontal support surface 180 has been positioned adjacent a surgical access site S, with the exemplary support surface comprising a small stand that can be placed over a leg of the patient P (with the legs of the stand straddling a leg of the patient). A guide catheter 182 is introduced into and advanced within the vasculature of the patient, optionally through an introducer sheath 184 (though no introducer sheath may be used in alternate embodiments). Guide catheter 182 may optionally have a single pull-wire for articulation of a distal portion of the guide catheter, similar to the guide catheter used with the MitraClip™ mitral valve therapy system as commercially available from Abbott. Alternatively, the guide catheter may be an unarticulated tubular structure, or use of the guide catheter may be avoided. Regardless, when used the guide catheter will often be advanced manually by the user toward a surgical site over a guidewire using conventional techniques, with the guide catheter often being advanced up the inferior vena cava (IVC) to the right atrium, and optionally through the septum into the left atrium.
As can be understood with reference to FIGS. 1, 10A, and 10B, driver assembly 14 may be placed on support surface 180, and the driver assembly may be slid along the support surface roughly into alignment with the guide catheter 182. A proximal housing of guide catheter 182 and/or an adjacent tubular guide catheter body can be releasably affixed to a catheter support 186 of stand 72, with the support typically allowing rotation and/or axial sliding of the guide catheter prior to full affixation (such as by tightening a clamp of the support).

As can be understood with reference to FIGS. 1, 10B, and 10C, catheter 12 can be advanced distally through guide catheter 182, with the user manually manipulating the catheter by grasping the catheter body and/or proximal housing 68. Note that the manipulation and advancement of the access wire, guide catheter, and catheter to this point may be performed manually so as to provide the user with the full benefit of tactile feedback and the like. As can be further understood with reference to FIGS. 1, 10C, and 10D, as the distal end of catheter 12 extends near, to, or from a distal end of the guide catheter into the treatment area adjacent the target tissue (such as into the left atrium) by a desired amount, the user can manually bring the catheter interface 120 down into engagement with the driver interface 94, preferably latching the catheter to the driver through the sterile junction as described above.

In methods that avoid the use of a guide catheter such as that shown affixed to a distal clamp of the stand by support 186, a guidewire (such as a super stiff guidewire or extra stiff guidewire) may instead be affixed to a guidewire support of the stand proximally of driver assembly 14, typically after catheter 12 is loaded retrograde onto the guidewire and is advanced over the guidewire to so that a distal end of the catheter is adjacent the target tissue (and so that the proximal housing of the catheter is distal of the proximal guidewire support or clamp). The stand may include both a distal releasable clamp or support 186 for the guide catheter (as shown) and a releasable proximal clamp or support for the guidewire proximal of the rails (not shown). Both the guide catheter clamp and guidewire clamp may be used together for some procedures, with the guidewire often ending proximally of (or having only a highly flexible distal portion extending into) the articulated portion of the catheter, which will often extend distally of (or be articulated distally of) the distal end of the guide catheter.

Referring now to FIGS. 1 and 10E, the driver and sterile housing will typically be in a relatively proximal axial position relative to the stand when the catheter engages the driver, so that the user can make use of the robotic articulation of the distal portion of the catheter during final advancement of the therapeutic tool of the catheter into alignment with the target tissue. Stand 72 may optionally have a holder for input 16. In some embodiments, the input may be used to enter articulation commands while supported by stand 72. The input can optionally be affixed to the stand or the sterile housing, or mounted to the driver and manipulatable by the user through a membrane of the sterile housing, or placed on support surface 180, or the like. As described above with reference to FIGS. 9A and 9B, the user may optionally perform a portion of the final distal advancement by sliding driver assembly 14 and catheter 12 along the rails of stand 72, with the processor deriving articulation commands for the distal articulated portion at least in part in response to signals from the axial signal sensor. Additional description of articulation commands may be found in, for example, Published US Application No. US-2017-0157361, entitled “Input and Articulation System for Catheters and Other Uses,” assigned to the assignee of the subject application and incorporated herein by reference. Optionally, at least a portion of the final advancement of the tool of the catheter may be performed by robotically articulating the catheter, including by elongating an articulated segment along the distal portion of the catheter as described above with reference to FIGS. 3A and 3B.

While the exemplary embodiments have been described in some detail for clarity of understanding and by way of example, a variety of modifications, changes, and adaptations of the structures and methods described herein will be obvious to those of skill in the art. Hence, the scope of the present invention is limited solely by the claims attached hereto.

1. A robotic system for treating a patient, the patient having a tissue accessible from within a sterile field, the system comprising:
an actuated tool having a proximal tool interface and a distal portion configured for alignment with the tissue, an actuatable feature disposed along the distal portion being operatively coupled with the tool interface;
a driver having a fluid supply configured to drive the tool and having a driver interface; and
a sterile housing fittingly receiving the driver, the sterile housing including a sterile junction having a sterile barrier extendable between the tool interface and the driver interface so that the fluid supply can drive the actuatable feature through the sterile housing when the tool interface is coupled with the driver interface, the sterile housing having an outer surface and maintaining sterile separation between the sterile field adjacent the outer surface and the driver within the sterile housing when the robotic surgical system is configured for use.

2. The system of claim 1, wherein the tool comprises an elongate flexible catheter body and the actuatable feature comprises an articulatable portion of the catheter body such that fluid from the fluid supply fluidically articulates the articulatable portion of the catheter body through the sterile housing.

3. The system of claim 2, wherein the articulatable portion comprises an articulation balloon array.

4. The system of claim 1, wherein the fluid supply comprises a disposable cartridge containing a pressurized mixture of gas and liquid, and wherein the driver comprises a plurality of valves and a processor configured to direct the gas from the cartridge along a plurality of fluid channels toward the driver interface.

5. The system of claim 4, wherein the canister comprises a frangible seal, wherein the receptacle is oriented, during use, to receive the canister with the frangible seal above the liquid so that un-vaporized liquid primarily remains within the canister and the fluid transmitted from the canister toward the driver interface primarily comprises the gas.

6. The system of claim 1, wherein the valves and the processor are contained in a driver housing, the outer surface of the driver housing configured to be wiped cleaned, between use on different patients, while the valves and processor remain within the driver housing.

7. The system of claim 6, wherein the driver housing is unarticulated during use and no motion of the tool is imparted by movement of a solid structure extending through the driver housing toward the tool.

8. The system of claim 1, wherein the sterile housing comprises a semi-rigid or rigid polymer shell having inner features that fittingly receive the driver so as to inhibit movement of the driver within the sterile housing, and wherein the sterile housing comprises a first housing portion with a first housing latch portion and a second housing portion with a second housing latch portion configured to releasable latch with the first latch portion so as to affix the housing portions together with the driver therein.

9. The system of claim 8, wherein the second housing portion comprises the sterile barrier of the sterile junction and is disposed above the first housing portion when the system is configured for use so that the tool is moved down to couple the tool interface with the driver interface, the sterile barrier extending around fluid passages of the sterile junction.

10. The system of claim 1, further comprising a tool latch releasably affixing the tool to the driver through the sterile junction, and a tool latch sensor transmitting signals in response to a state of the tool latch, the system configured to inhibit applying fluid pressure from the fluid source to the driver interface when the sensor signals indicate the tool is not safely latched to the driver.

11. The system of claim 1, wherein the tool comprises an elongate body having an axis extending between the proximal interface and the distal portion, and further comprising a stand supporting the sterile housing so that the sterile housing moves along the axis.

12. The system of claim 11, further comprising an input for receiving a movement command and a processor coupling the input with the fluid source so that fluid from the fluid source induces movement of the tool per the movement command, and an axial position sensor coupled with the stand and configured to transmit axial position signals to the processor in response to an axial position of the sterile housing relative to the stand

13. A sterile structure for use in a robotic surgical system for treating a patient, the patient having a tissue accessible from within a sterile field, the system including:
an actuated tool having a proximal tool interface and a distal portion configured for engaging the tissue, an actuatable feature disposed along the distal portion being operatively coupled with the tool interface; and
a driver configured to drive the tool and having a driver interface;
the sterile structure comprising:
a sterile housing having a receptacle configured to fittingly receive the driver, the sterile housing including a sterile junction having a sterile barrier extendable between the tool interface and the driver interface so that the driver can drive the actuatable feature through the sterile housing when the tool interface is coupled with the driver interface, the sterile housing having an outer surface and maintaining sterile separation between the sterile field adjacent the outer surface and the driver within the sterile housing when the robotic surgical system is configured for use.

14. A sterile interface for use in a catheter system for treating a patient disposed in a sterile field, the system comprising:
an elongate flexible catheter body having a proximal catheter interface and a distal portion with an axis therebetween, a fluid-actuated feature disposed along the distal portion and a lumen system providing fluid communication between the fluid-actuated feature and a plurality of catheter fluid receptacles of the catheter interface; and
a driver assembly having a fluid supply and a driver interface with a plurality of driver fluid receptacles;
the sterile interface comprising:
a sterile junction having a sterile barrier body with a first surface and a second surface opposed to the first surface;
a plurality of tubular bodies traversing the sterile body, the tubular bodies having lumenal axes extending between first ends adjacent the first surface and second ends adjacent the second surface, the tubular bodies supported by the sterile barrier body with the axes aligned to facilitate detachably sealed fluid communication between the fluid supply and the fluid-actuated feature, and such that the axes can float so as to accommodate a tolerance of the fluid receptacles.

15. The sterile interface of claim 14, wherein a resilient sealing surface adjacent the ends of the tubular bodies facilitates sealing with the driver fluid receptacles and the catheter fluid receptacles, wherein a first feature protrudes radially from each of the tubular bodies adjacent the first surface and a second feature protrudes radially from each of the tubular bodies adjacent the second surface, the tubular bodies having profiles and extending through apertures in the sterile barrier body, the apertures being larger than the profiles and the sterile barrier body captured between the first and second features so that the tubular bodies can float laterally throughout a lateral tolerance range; and
further comprising a latch tension member extending rotatably through the sterile barrier body, the latch tension member comprising a first latch element adjacent the first surface, the first latch element configured to rotatably engage a driver latch feature of the driver so as to affix the sterile barrier body to the driver, the latch tension member comprising a second latch element adjacent the second surface, the second latch element configured to be rotatably engaged by a rotating element of the catheter interface so as to affix the sterile barrier body to the catheter, at least one of the latch elements and associated features comprising a cam-and-follower so that the latch tension member is in tension when a latch sensor generates signals indicating the catheter interface is rotatingly latched to the driver.

16.-23. (canceled)
